(19) United States
(12) Patent Application Publication
(10) Pub. No.: US 2025/0010051 A1
(43) Pub. Date: Jan. 9, 2025

(54) IMPLANT SYRINGE

(71) Applicant: Gaplast GmbH, Altenau (DE)

(72) Inventors: Stephan Kneer, Farchant (DE); Roland Kneer, Farchant (DE); Walter Koller, Unterammergau (DE)

(21) Appl. No.: 18/625,489

(22) Filed: Apr. 3, 2024

(30) Foreign Application Priority Data
Jul. 4, 2023 (DE) 10 2023 117 652.8

(51) Int. Cl. A61M 37/00; A61M 5/315

(52) U.S. Cl. CPC A61M 37/0069; A61M 5/31501; A61M 5/31511; A61M 2210/1021

(57) ABSTRACT

An implant syringe is provided and includes a cannula, a storage element for receiving a solid material implant, an outer sleeve with a radially projecting front gripping section, with which the cannula, the storage element and the outer sleeve are movable axially together, an inner sleeve on which the outer sleeve sits, where the outer sleeve is retractable by the front gripping section on the inner sleeve so that the cannula completely enters into the inner sleeve and where the inner sleeve includes a front needle guard and a rear spacer, which are locked together, where locking windows are formed in two opposing lugs on the needle guard, and the spacer has two locking hooks, so that to connect the two components, the lugs slide over the locking hooks on the spacer until the locking hooks lock into the locking windows.

### PRIORITY CLAIM

The subject application claims convention priority to German patent application No. DE 10 2023 117 652.8, filed Jul. 4, 2023.

### BACKGROUND

The invention relates to an implant syringe which is used to deliver a strand-shaped solid material implant with a long-term active substance into the body of a patient. The long-term preparation is generally placed in the abdominal wall of a patient, into which previously a receiving passage for the solid material implant was pierced by means of a cannula.

The invention starts from an implant syringe, which includes the following features: a cannula, a storage element for receiving a solid material implant, an outer sleeve with a radially projecting front gripping section, with which the cannula is axially movable together with the storage element and the outer sleeve, and further an inner sleeve, on which the outer sleeve sits and in which the cannula is slidable with the storage element, wherein the inner sleeve has axial slits, through which radial webs on the outer sleeve engage, wherein the implant syringe further has a rod-shaped piston, which is fastened to a rear gripping section and is partially surrounded by a rear sleeve, which can engage slidably in the inner sleeve, wherein the rod-shaped piston may be advanced through the storage element into the cannula so far that a gap remains between the tip of the cannula and the head end of the piston, which is approximately equal to the length of the solid material implant to be delivered, wherein, in the advanced starting position with the cannula exposed, the outer sleeve is releasably locked by a blocking device on the inner sleeve and when the rear sleeve is advanced into its end position the blocking device is moved into the released position, whereby the outer sleeve is retractable by means of the front gripping section on the inner sleeve so far that the cannula completely enters into the inner sleeve.

Such an implant syringe is disclosed in DE102017007893A1. In this implant syringe, the outer sleeve is connected integrally by means of radial webs to a needle holder, into the rear side of which the storage element, which receives the solid material implant, is inserted. After the assembly of the inner sleeve and outer sleeve, the cannula is secured by adhesive in a front opening in the needle holder. The solid material implant is inserted into the storage element through the open rear side of the inner sleeve and is advanced by the rod-shaped piston in the storage element into the lightly clamped starting position. When using the implant syringe to deliver the solid material implant into a patient, the rear gripping section is then advanced with the piston until the solid material implant has been advanced to the tip of the cannula, whereafter the outer sleeve, which is exposed in this position, is retracted with the cannula and the storage element whilst leaving the solid material implant behind in the body of the patient.

An implant syringe is known from DE102020122654A1, in which the cannula is firmly connected to the storage element to form a separate, that is to say separated from the remainder of the implant syringe, cannula unit, whereby the insertion of a solid material implant into the storage element is considerably simplified. The invention provides further that the webs on the outer sleeve are attached at their radially inner ends to an annular receiving member, which is situated within the inner sleeve, and that the cannula unit is insertable with the solid material implant inserted into the storage element into the receiving member and is lockable therein.

In these known implant syringes, the inner sleeve is composed of two components, namely the front needle guard and the rear spacer, which are locked together. For this purpose, two locking hooks are formed on lang webs an the needle guard, which snap into locking windows in the spacer.

When tensional forces act on this locking connection, the locking hooks are stretched, whereby, determined by the component, only a very little space is available for the locking hooks. With a cross-section determined by the construction of preferably only ca. 1.5 mm². the locking hooks withstand only a tensional force of ca. 10N. However, a higher retaining force of the snap connection and thus a more stable construction of the implant syringe is desirable. Since, determined by the component, no more space for locking hooks with a greater cross-section is available, a solution to the problem by locking hooks with a greater cross-section cannot be considered without impairing the handleability and the optical appearance of the implant syringe.

It is thus the object of the invention to provide a better locking connection with which the retaining force is considerably increased.

### BACKGROUND

The invention provides that a respective locking window is formed in two diametrically opposed lugs on the needle guard member and that the spacer affords two locking hooks at corresponding positions. When snap connecting the two components, the lugs slide over the locking hooks on the spacer until the locking hooks lock into the locking windows in the lugs. The end sections of the two lugs on the needle guard are thus received flush in recesses in the spacer. A stable, unreleasable snap connection on both sides is thus produced, in which, when attempting to separate the two components by pulling them apart, the applied force is no longer absorbed by stretching of the plastic material but by pressure on the plastic material. The retaining force of the connection is hereby significantly increased without the outer circumference of the inner sleeve being increased and its optical appearance being significantly altered.

In an advantageous embodiment of the invention, the two lugs on the needle holder, which afford the locking windows, have a different length so that, with a corresponding construction of the two locking hook regions, only a single assembly position of the two components is possible.

The locking hooks preferably have a massive wedge shape with an upper side rising obliquely in the assembly direction and a rear side, which is preferably undercut at a flat angle so that the snap connection is practically unreleasable. The end sections of the lugs should engage flush in recesses in the spacer.

### BRIEF DESCRIPTION OF THE DRAWINGS

FIG. 1a is a perspective view of one embodiment of the implant syringe;

FIG. 1b is a cross-sectional view of the implant syringe of FIG. 1a taken substantially along the line 1b-1b in the direction generally indicated;

FIG. 2a is a perspective view of the rear sleeve of the implant syringe of FIG. 1.

FIG. 2b is a cross-sectional view of the rear sleeve of the implant syringe of FIG. 2a taken substantially along line 2b-2b in the direction generally indicated.

FIG. 3 is a perspective view of the inner sleeve of the implant syringe of FIG. 1 composed of the needle guard member and spacer member;

FIG. 4 is a fragmentary cross-sectional view of the needle guard member of the inner sleeve of FIG. 3;

FIG. 5 is a cross-sectional view of the spacer member of the inner sleeve of FIG. 3;

FIG. 6 is a perspective of another embodiment of the inner sleeve in accordance with the invention;

FIG. 7 is an exploded perspective view of the needle guard and the spacer; and

FIG. 8 is a fragmentary cross-sectional view of the inner sleeve in a locked state.

### DETAILED DESCRIPTION

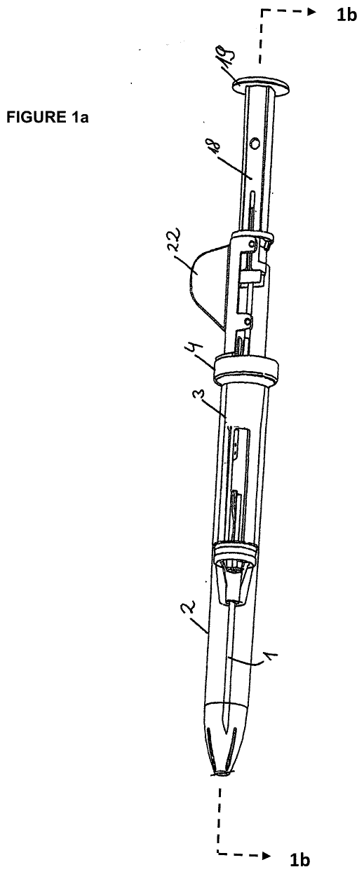

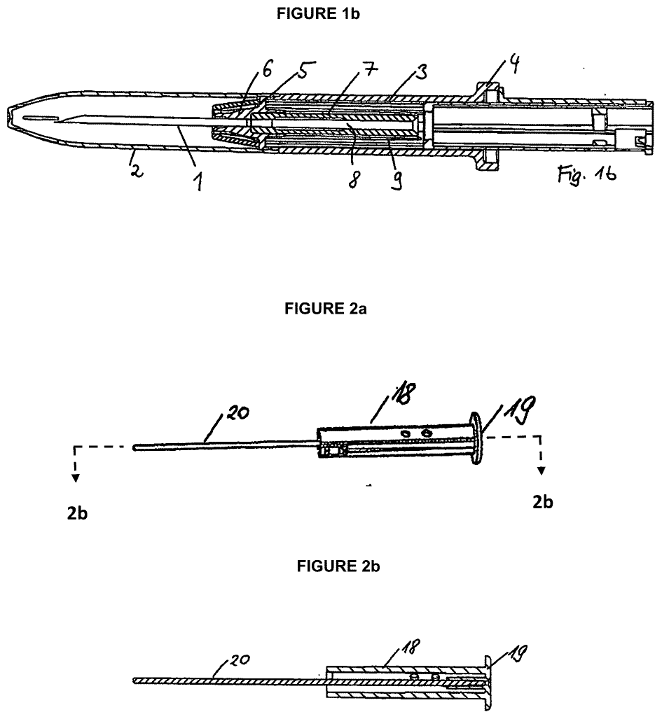

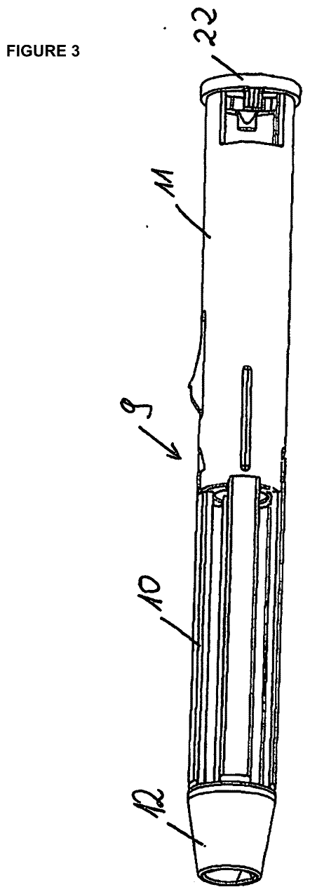

FIGS. 1a and 1b show an initial state of the implant syringe, in which the syringe needle 1 is covered by a removable protective cap 2. The removable protective cap 2 is lightly, releasably locked to an outer sleeve 3. The outer sleeve 3 has an elongate cylindrical form, wherein integrally formed at its end section, on the right in the figures, there is a radially projecting front gripping section 4. The outer sleeve 3 is firmly connected by means of a plurality of radially inwardly extending webs 5 to a needle holder 6, in which the syringe needle 1 is secured. Axially behind the needle holder 6 a preparation receiving member 7 is so arranged that its passage 8 is aligned with the passage in the syringe needle 1. Located in this preparation receiving member 8 there is preparation, not shown, which, when the implant syringe is used, is deposited e.g. in the abdominal wall of a patient.

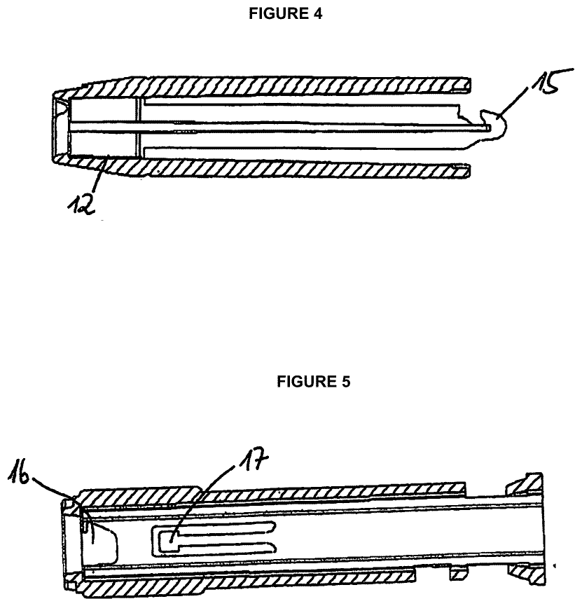

The outer sleeve 3 is located (when it is not blocked) slidably on an inner sleeve 9, which is composed of a front needle guard 10 and a rear spacer 11. The needle guard 10 includes a front, conically tapering section 12, which is closed in the peripheral direction and covers the tip of the completely retracted syringe needle 1. The axially adjoining region includes webs or lugs 13 spaced in the peripheral direction, wherein in the prior art hooks 15 are formed on the end sections of two webs, which lock into recesses in the spacer 11 of the inner sleeve 9 in order to connect the two components (FIGS. 4 and 5).

The rear portion 11 of the inner sleeve 9 includes a spring arm 17, which is cut away from the wall of the inner sleeve member 11 and projects radially outwardly with a locking hook. This spring arm 17 with the locking hook serves as a blocking device for the outer sleeve 3 so long as a rear sleeve 18 is not slid into its end position in the rear portion 11 of the inner sleeve 9.

Integrally formed on the rear end of the rear sleeve 18 is a flat gripping member 19, fastened to which centrally in the sleeve 18 there is a rod-shaped piston 20. The rear sleeve 18 includes a slit, which extends in the axial direction and which when the rear sleeve 18 is slid into the rear spacer 11 of the inner sleeve cooperates with the spring arm 17 by means of an oblique surface so that its locking hook is drawn radially inwardly. This is the case when the inner side of the rear gripping member 19 abuts the rear edge 22 of the inner sleeve 9, wherein this position is lightly releasably locked.

The outer sleeve 3 can now be retracted together with the syringe needle into its end position, in which the two gripping members 4 and 19 engage one another. The tip of the injection needle 1 is now located within the frustoconical section 12 of the inner sleeve 9.

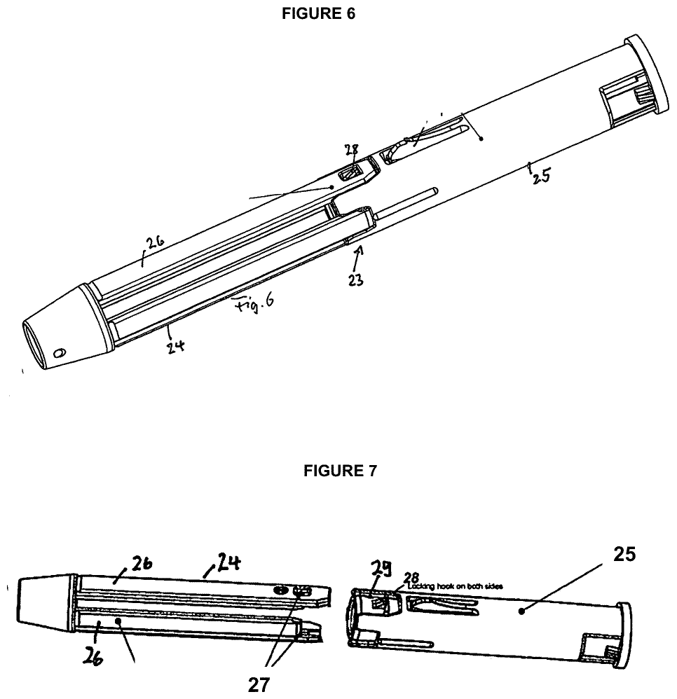

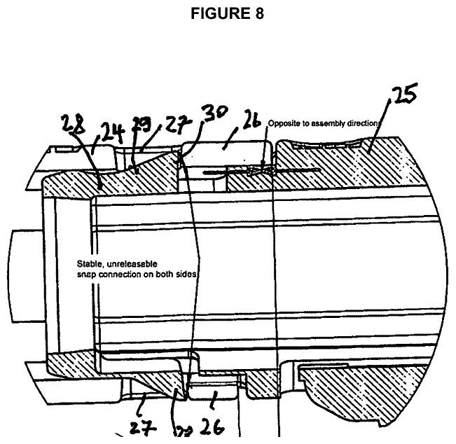

FIGS. 6 to 8 show an embodiment of the locking in accordance with the invention of the inner sleeve 23, which is composed of the front needle guard 24 and the rear spacer 25. The needle guard 24 includes two elongate lugs 26, formed in the end region of which are two locking windows 27. Formed on the spacer 25 at corresponding positions are two locking hooks 28, which snap into the locking windows 27 when the needle guard 27 is put together with the spacer 25. The end regions of the lugs 26, which overlap with the spacer 25, move flush into recesses 29 in the spacer 25.

The locking hooks 28 have a massive structure, which is approximately wedge-shaped. The upper side 29 of the locking hooks rises obliquely upwardly in the assembly direction whilst the rear side 30 of the locking hooks 28 is undercut at a flat angle. The snap connection is thereby stable and practically unreleasable.

The two lugs 26 with the locking windows 27 have a different length, whereby the two associated regions of the locking windows are correspondingly positioned so that only one assembly position is possible.

1. An implant syringe with a cannula, a storage element for receiving a solid material implant, an outer sleeve with a radially projecting front gripping section, with which the cannula, the storage element and the outer sleeve are movable axially together, an inner sleeve, on which the outer sleeve sits and in which the cannula is slidable with the storage element, wherein the inner sleeve has axial slits, through which radial webs on the outer sleeve engage, further with a rod-shaped piston, which is secured to a rear gripping section and is partially surrounded by a rear sleeve, which can engage slidably in the inner sleeve, wherein the rod-shaped piston may be advanced through the storage element into the cannula so far that a gap remains between the tip of the cannula and the head end of the piston which is approximately equal to the length of the solid material implant to be delivered, wherein the outer sleeve is releasably locked in the advanced starting position with the cannula exposed by a blocking device on the inner sleeve and the blocking device is moved, when the rear sleeve is advanced into its end position, into the released position, whereby the outer sleeve is retractable by means of the front gripping section on the inner sleeve so that the cannula completely enters into the inner sleeve and wherein the inner sleeve is composed of a front needle guard and a rear spacer, which are locked together, wherein locking windows are formed in two opposing lugs on the needle guard, and wherein that the spacer has two locking hooks at corresponding positions and that to connect the two components, the lugs slide over the locking hooks on the spacer until the locking hooks lock into the locking windows.

2. The implant syringe as claimed in claim 1, wherein in the snap connection, end sections of the lugs engage flush in recesses in the spacer.

3. The implant syringe as claimed in claim 1, wherein the locking hooks have a substantially massive wedge shape with an upper side, which rises obliquely in the assembly direction, and a rear side, which is undercut at a flat angle so that the snap connection is unreleasable.

4. The implant syringe as claimed in claim 1, wherein the two lugs have a different length and the two regions of the locking windows are correspondingly constructed so that only one assembly position is possible.
